(19) United States
(12) Patent Application Publication
(10) Pub. No.: US 2022/0163506 A1
(43) Pub. Date: May 26, 2022

(54) Processing System for Measuring and/or Processing Measured Pressure and/or Humidity Values

(71) Applicant: B-Horizon GmbH, Sinzing (DE)

(72) Inventor: Mohammad Kabany, Regensburg (DE)

(21) Appl. No.: 17/442,253

(22) PCT Filed: Mar. 27, 2020
(86) PCT No.: PCT/EP2020/058723

§ 371 Date: Sep. 23, 2021

(30) Foreign Application Priority Data
Mar. 29, 2019 (DE) 10 2019 108 185.8

(51) Int. Cl. G01N 33/36; H04Q 9/00; A41D 13/12

(52) U.S. Cl. CPC G01N 33/367; H04Q 2209/40; A41D 13/1281; H04Q 9/00

(57) ABSTRACT

The invention relates to a sensor system for measuring and/or processing measured pressure and/or humidity values, comprising at least one sensor for measuring pressure and/or humidity and at least one processing unit, which is set up and intended to control the sensor and/or to store and/or process data measured by the sensor. Furthermore, the sensor system comprises at least one evaluation unit, which evaluates the data forwarded to it by the processing unit and subsequently forwards these data or a data record generated from the data to a CPU, in particular wirelessly, wherein these data comprise a user behavior, movement sequences, body functions, body behavior, weight, pressure or moisture of a skin of the user, and are collected and subsequently evaluated by the CPU.

The present invention relates to a processing system for measuring and/or processing measured pressure and/or moisture values, in particular on articles of clothing, and to a sensor arrangement and an object of use in accordance with the respective generic terms of claims 1, 7 and 8.

The processing system proposed here comprises at least one sensor for measuring pressure and/or humidity. The sensor may be based on a mechanical, thermoelectric, resistive, piezoelectric, capacitive, inductive, optical and/or magnetic principle.

For example, the sensor proposed here may be a pressure sensor and/or a humidity sensor. In particular, the sensor can also be a tilt sensor, a force sensor, a CCD sensor, a photocell, a Hall sensor or a thermocouple.

Different active principles can also be combined with each other, so that a sensor is created which, for example, is based both on capacitive active principles and on an optical active principle.

According to at least one embodiment, the processing system comprises at least one processing unit which is set up and intended to control the sensor and/or to store and/or process data measured by the sensor.

Furthermore, the sensor system comprises at least one evaluation unit, which evaluates the data forwarded to it by the processing unit in order to subsequently forward this data or a data record generated from the data to a CPU, in particular wirelessly.

In this context, the CPU may be part of the processing system.

According to the invention, at least the sensor, but preferably also the processing unit, is arranged on a flexible and bendable carrier material, wherein the carrier material is adapted and intended to be arranged on a surface of an object of use, in particular on the surface of a piece of clothing, in particular wherein these data comprise a user behavior, movement sequences, body functions, body behavior, weight, pressure or moisture of a skin of the user, and is collected and subsequently evaluated by the CPU.

The object of use may be items of clothing, as “wearables”.

Previously known carrier materials are neither flexible nor bendable, but for example in the form of an adhesive layer and/or a substrate. However, it is now proposed here to offer, instead of such a non-flexible and non-bendable carrier material, one which has just flexible and bendable properties.

“Flexible” in this context means that the carrier material is bendable and thus elastic at least in places.

The carrier material therefore adapts to the shape of the object of use, for example to the shape of the garment. Preferably, the carrier material is arranged on the surface of the utilization object directly or indirectly. In the case of direct arrangement, the carrier material is in direct contact with the surface of the usage object.

According to at least one embodiment, the sensor system for measuring and/or processing measured pressure and/or humidity values comprises at least one sensor for measuring pressure and/or humidity and at least one processing unit, which is arranged and provided for controlling the sensor and/or for storing and/or processing data measured by the sensor.

Furthermore, the sensor system comprises at least one evaluation unit which evaluates the data forwarded to it by the processing unit and then forwards these data or a data record generated from the data to a CPU, in particular wirelessly, wherein according to the invention at least the sensor, but preferably also the processing unit, are arranged on a flexible and bendable carrier material, wherein the carrier material is adapted and intended to be arranged on a surface of an object of use, in particular on the surface of a piece of clothing, in particular wherein these data comprise a user behavior, movement sequences, body functions, body behavior, weight, pressure or moisture of a skin of the user, and are collected and subsequently evaluated by the CPU.

According to at least one embodiment, the carrier material is a textile material, in particular a woven material and/or a leather material and/or a plastic material.

In the sense of the invention, a woven fabric is therefore a fabric that has been woven manually or mechanically on the basis of individual threads.

For example, electrical conductor tracks are woven into the woven fabric for electrical contacting of the sensor and the processing unit. The electrical conductor paths can therefore be integrated in the woven fabric in addition to the usual fibers and woven fabric strands, or they can replace individual woven fabric strands that form the woven fabric network.

These conductive traces may therefore provide an electrical connection between the sensor and the processing unit. Also, the conductive traces may form an electrical connection between a battery or other power generating element between the sensor and/or the processing element.

Depending on the spacing and properties of the individual yarns (high twist, puffy, etc.), quite loose fabrics, such as a bandage fabric or dense fabric such as brocade fabric, can be formed, as longitudinally elastic fabrics are used by rubber threads used as chain threads (more tapes), or crimping and/or bulking yarns, they are tensioned, processed and contract when at rest. Bulk yarns consist of textured, i.e. crimped, synthetic fibers. The crimping changes the properties of the synthetic fibers. The yarns spun on them are very elastic and voluminous and have good thermal insulation properties.

According to at least one embodiment, the carrier material is in the form of a carrier strip, so that the sensor and the processing unit are arranged one behind the other along the main strip extension.

The “strip main extension” is that length dimension and length direction of the carrier material which represents a main extension direction. Compared to the length of the carrier material in the direction of the main strip extension, a width of the carrier material is therefore negligible.

According to at least one embodiment, the evaluation comprises a representation of a temporal usage of the usage object, a representation of the temporal movement pattern of the usage object a representation of the temporal pattern of pressure and/or humidity of a skin of the user. This representation can be taken over by the CPU, which has, for example, a screen on which a duration of use, a temporal intensity of use or the like can be graphically displayed.

According to at least one embodiment, the CPU, the evaluation unit and/or the processing unit is installed in a mobile terminal, in particular in a smartphone.

According to at least one embodiment, the evaluation comprises a suggested action for the user for the future use of the utilization object.

According to at least one embodiment, the utilization object is a textile material, which is incorporated in a shoe, a shirt or another garment of the user.

According to at least one embodiment, the CPU makes at least one usage suggestion, in particular with regard to a future temporal usage of the usage object, wherein the user confirms, changes and/or rejects the usage suggestion, and further wherein in case of a change and/or a rejection of the usage suggestion by the user, the CPU adapts a then changed user profile to then changed user data. A user profile in the sense of the invention may be a compilation of characteristic properties, capabilities and/or requirements of a user.

Furthermore, the present invention relates to a sensor arrangement for measuring and/or processing measured pressure and/or humidity values.

The described sensor arrangement comprises at least one sensor for measuring pressure and/or humidity as well as at least one processing unit, which is set up and intended to control the sensor and/or to store and/or process data measured by the sensor.

At least the sensor, but preferably also the processing unit, are arranged on a flexible and bendable carrier material, which carrier material is adapted and intended to be arranged on the surface of the one utilization object.

The sensor arrangement proposed herein comprises the same advantageous embodiments and advantages as the sensor system described above.

The sensor system differs from the sensor arrangement in particular in that the sensor system additionally comprises the described CPU.

The sensor and/or the processing unit and/or the CPU can be supplied with electrical energy by means of a battery or a fixed power supply.

Alternatively or additionally, the generation of electrical energy to supply the sensor and/or processing unit is possible by means of so-called “energy harvesting”.

Energy harvesting refers to the extraction of small amounts of electrical energy from sources such as ambient temperature, vibrations or air currents for low-power mobile devices. The structures used for this purpose are also known as nanogenerators. Energy harvesting avoids limitations of wired power or batteries for wireless technologies.

Energy Harvesting Options:
Piezoelectric crystals generate electrical voltages when force is applied, for example by pressure or vibration. These crystals can be arranged on or on the carrier material;
Thermoelectric generators and pyroelectric crystals generate electrical energy from temperature differences. These generators can be arranged on or on the carrier material;
   Via antennas, the energy of radio waves, a form of electromagnetic radiation, can be captured and used energetically. Passive RFIDs are an example of this. These antennas can be arranged on or on the carrier material;
   Photovoltaics, electrical energy from ambient lighting;
   Osmosis.

For example, such an energy storage and/or energy generation element is also arranged on the carrier material to supply the sensor and/or the processing unit with electrical energy.

An energy storage device of the device may be part of a processing unit. To this end, one or more of the processing units may include such an energy storage device (local energy storage device). For example, only one or some of the processing units have such an energy storage, so that one of these processing units supplies another processing unit (namely one which does not have an energy storage) with electrical energy.

It is also conceivable that the energy storage unit(s) of the processing unit(s) supplies the CPU with electrical energy in whole or in part. For example, the CPU may thus be connected to no further energy storage and/or energy supply lines.

Via the aforementioned energy harvesting, at least one of the energy storage units can be charged.

The energy transfer between the sensors and/or the processing units and/or the CPU can be completely or partially wireless.

Wireless power transmission in the near field, also referred to as non-radiative coupling, includes, for example, inductive coupling based on magnetic flux. Often, the term wireless power transmission is used synonymously with inductive power transmission, since the latter plays a dominant role in practical applications. Wave phenomena do not play a role in nonradiative coupling in the near field.

For example, wireless energy transfer between elements occurs by means of inductive coupling, resonant inductive coupling, and/or capacitive coupling.

For example, the sensor is constructed as follows and comprises at least one capacitor with at least two electrodes, which are arranged, in particular in a horizontal direction along and on a, in particular the, in particular flexible, carrier material with respect to each other, wherein at least one dielectric layer is arranged between the electrodes.

The sensor is characterized in that on a side of at least one electrode facing away from the carrier material and/or of the dielectric layer at least one at least partially liquid-permeable and/or liquid-absorbing moisture layer is arranged at least in places, the at least one electrode and/or the dielectric layer thus being arranged in a transverse direction between the carrier material and the moisture layer, such that a capacitance is at least partially changed by the liquid at least partially impinging on the dielectric layer, wherein a processing unit is to arranged and provided for measuring and/or storing said change, such that a capacitive moisture sensor is formed.

For example, in a tire, two or more processing units and/or two or more processing units of different tires communicate with each other.

Conceivably, the plurality of processing units form a processing network, wherein the acquisition, processing and/or communication of the sensor data and/or the processing data of each sensor and/or processing unit is controlled by at least one control unit (master). The control unit may be identical to the CPU described above.

However, it is also possible that one or more of the processing units represent the master, which controls the remaining processing units (slave) and/or the remaining sensors (slave).

For example, one of the processing units and/or the CPU may, after the device has been put into operation (for example, after the device has been switched on), select those sensors which are put into operation for a predetermined period of use. Alternatively, all or some sensors may be put into operation, but then it is conceivable that one of the processing units and/or the CPU, in particular for the purpose of energy saving, only forwards data of a predetermined number (i.e. less than all sensors) of sensors to the CPU (filtering).

This master processing unit can preferably communicate with the CPU as a single unit.

Further alternatively or additionally it is conceivable that one or all processing units and/or a sensor (slave or master) communicate directly with the CPU.

According to at least one embodiment, the processing network can be subdivided by means of at least one VLAN switch into at least two network segments (VLANs) that are only logically separated from one another, and wherein each of the sensing elements can be controlled as a function of the control by a VLAN switch and/or the control device and thus by each of the network segments.

If, for example, a very large area (for example, a textile) is equipped with a plurality of sensors and processing units claimed here, individual processing units and/or sensors can then be categorized (according to different priorities, etc.) in a particularly simple manner. Thus, in one embodiment, a “virtual”, i.e. VLAN subdivision is selected instead of a physical network subdivision. This ensures that changes in the categorization of the processing units and/or sensors can be reacted to particularly quickly and without time-consuming conversion work.

According to at least one embodiment, the control system comprises at least one processing network, wherein by means of at least one VLAN switch of the processing network, the latter can be subdivided into at least two network segments (VLAN) which are only logically separated from one another, and wherein each processing unit and/or each of the sensors can be controlled by each of the network segments depending on the control by the VLAN switch.

For this purpose, the VLAN switch may be installed in at least one of the processing units and/or sensors or in a separate component.

According to at least one embodiment, prioritization of the individual network segments is performed by means of the VLAN switch, in particular with regard to their data exchange.

According to at least one embodiment, at least one VLAN ID is assigned to each processing unit and/or each network segment, wherein at least one sensor or another processing unit can be controlled via each of the VLAN IDs. Individual sensors and/or individual processing units can form their own subnetwork.

To communicate across network boundaries, the state of the art uses static project dynamic routes. This model of separation is clear and concise and has been used for years. However, this has the disadvantage that broadcast requests on the subnet are visible to all subscribers and would have to be viewed by the endpoints. In other words, different endpoints could previously only be controlled via corresponding switches assigned to each subnet, which were separate and physically separated from each other. However, such a setup is particularly costly and extensive in design.

As mentioned above, the design of each subnetwork with a separate switch and separate physical data lines is thus dispensed with in particular, so that a single physical structure can be applied for the entire network, whereby this physical structure, i.e. network architecture, is only separated on the basis of a logical, in particular also mathematical distinction (i.e. thought).

In this context, the abbreviation “VLAN switch” refers to such a network switch which is set up and intended to operate a network in the form of a Virtual Local Area Network (VLAN).

In this respect, the network segments now claimed, which can each be designed in the form of a VLAN network, thus make it possible for the separation of the network to be divided into several logical segments, i.e. the network segments.

Unlike physical separation by assignment to a switch port, separation by VLANs logically separates the devices by a VLAN ID. In this process, the data stream of each station is provided with an identifier (the VLAN “tag”). This identifier determines the affiliation of a data packet to a specific VLAN. All devices with the same VLAN ID are now in one logical network.

In particular, a broadcast can be limited by the logical separation of the individual networks. Broadcasts are distributed only to members of the same VLAN and not to all control elements connected to the switch.

In this respect, this also contributes not only to higher performance, but also to greater security, because the data traffic is restricted to fewer addressees. In addition, users or the control elements in a VLAN usually have no possibility of breaking out of the assigned VLAN. Access (or attack) to another computer that does not belong to its own VLAN can therefore already be prevented by the network switch. If cross-VLAN communication is necessary, routes can be explicitly set up for this purpose.

In particular, it is noted that the VLAN technology described herein may be one that is adapted to and/or compatible with the IEEE 802.1Q industry standard.

The IEEE 802.1Q standard is a prioritization and VLAN technology standardized by the IEEE, which implements packet-based tagged VLANs in contrast to the older, only port-based VLANs. The term “tagged” is derived from the English expression “material tags”.

Tagged VLANs are therefore networks that use network packets that carry a special VLAN tag.

In particular, data fields for V-LAN tagging are defined in the 802.1Q standard, which can be introduced in the data area of an Ethernet packet.

In this respect, the present network can be designed in the form of an Ethernet communication system.

This has the advantage that, as a rule, existing, older switches can also forward such packets. The inserted tag usually consists of several fields, for example four fields with a total length of 32 bits.

For the protocol ID 2 bytes are used, for the priority field 3 bits, for indicator of the canonical format 1 bit and for VLAN ID 12 bits.

To uniquely identify a VLAN, each VLAN is therefore first assigned a unique number. This number is called the VLAN ID. A detection module equipped with VLAN ID=1 can communicate with any other device in the same VLAN, but not with a device in a different VLAN, such as ID=2, 3, . . .

To distinguish between VLANS, the IEEE 802.1Q standard adds 4 bytes to an Ethernet frame. Of these, 12 bits are provided to accommodate the VLAN ID, so that (without using the Canonical format) 4096−2=4094 VLANS are theoretically possible.

It is conceivable that the individual logical network connections are designed according to an OPC standard, i.e., for example, in the form of OPC UA connections. In particular, it is conceivable that several OPC UA end points with different IP address, VLAN ID and prioritization are available per network segment via the control device in accordance with the IEEE 802.1Q standard mentioned above.

If a network segment which has been uniquely, preferably uniquely, assigned a specific VLAN ID has a higher priority than a network segment which differs from it only logically and has a correspondingly different VLAN ID, the control device and/or the VLAN switch can be designed to give priority to the data exchange of the higher-priority network segment first in order to allow the lower-priority network segment to be processed only after the tasks assigned to this higher-priority network segment have been completed.

In other words, the following generally applies: assignment and configuration of the OPC UA endpoints to a specific network segment according to the VLAN ID and assignment of a priority according to the priority of the corresponding VLAN.

According to at least one embodiment, each sensor and/or each processing unit is assigned at least one VLAN ID and each network segment is in turn assigned at least one, for example exactly one, unique, preferably one-to-one, VLAN ID, wherein at least one control element can be controlled via each of the VLAN IDs. According to at least one embodiment, at least one device comprises at least one temperature sensor, wherein the temperature sensor measures an ambient temperature and/or a temperature of a sensor and forwards it to the processing unit of a device and/or to the central CPU.

According to at least one embodiment, the central CPU determines a degree of utilization (CPU load and/or memory consumption) of at least one processing unit, whereby if a limit temperature of the processing unit and/or at least of the sensor assigned to this processing unit is exceeded, this/these is/are at least partially throttled or completely switched off.

Besides, it is additionally or alternatively conceivable that the evaluation unit(s) communicate with each other and are interconnected in the same way as the processing unit in the sense of the above network.

In addition, the present invention also comprises an object of use for use by a user, in particular using the above processing system.

In particular, this usage object can be the usage object depicted above, wherein at least one sensor for measuring pressure and/or moisture is arranged on a surface of the usage object, and wherein at least the sensor is arranged on or under a, in particular flexible and bendable, carrier material of the usage object, wherein the carrier material is arranged on a surface of a usage object, in particular on the surface of a piece of clothing, wherein the sensor measures these data a user behavior, movement sequences, body functions, body behavior, weight, pressure or moisture of a skin of the user, wherein the sensor is set up for this purpose and is provided for sending these data subsequently to a CPU, so that these data are then subsequently evaluated by CPU.

According to at least one embodiment, the sensor is embedded in a shoe, in particular in a glove, or other piece of clothing, in order to collect a movement profile, a movement intensity and/or a movement duration, the data then being sent to the CPU, which may be built into a game console, a smartphone and/or a PC. This data can be used for navigation and/or for generating sports profiles.

In the following, the invention is described in more detail with reference to an example of an embodiment and the associated FIGURES.

FIG. 1 shows an embodiment of a sectional view of an usage object 100 proposed herein, in this case a shoe, within which the arrangements proposed in this application are at least partially arranged.

Components that are the same or have the same effect are provided with the same reference signs, even if some reference signs or some components are shown in an exaggeratedly large size.

In FIG. 1, as mentioned above, a side view of an usage object 100 proposed herein, presently a shoe, is shown, wherein within the usage object 100 at least a part of a processing system 2000 for measuring and/or processing pressure and/or humidity values is shown.

It can be seen that a sensor arrangement 1000 comprises a plurality of sensors 1 for measuring pressure and/or moisture, wherein a preferably single processing unit 2 is provided for these sensors 1, which processing unit 2 is set up and intended to control the sensors 1 and/or to store and process data measured by the sensors 1.

Both the sensors 1 and the processing unit 2 are arranged on a flexible carrier material 11, the carrier material 11 being adapted and provided to be arranged on the surface of an object of use, in this case on an inner surface of the shoe 100.

Along the inner surface of the shoe 100, the carrier material 11 is formed in the form of a carrier strip, so that the sensors 1 and the processing unit 2 are arranged one behind the other along the main extension of the strip.

The carrier strip on which the sensors 1 and the processing unit 2 are arranged is formed in FIG. 1 by a central strip, which can be surrounded to the left and right by further optional carrier strips of the same type as the central carrier strip.

In addition, it is shown that another optional carrier strip may be arranged on an inner surface of the side wall of the shoe 100. This carrier strip may also be constructed in the same manner as described above. For example, it is also possible that only one carrier strip is arranged inside the tire 100 on an inner surface of the tire 100.

The processing system 2000 includes a CPU 4 external to the usage object 100.

For example, based on the processing of the data, the CPU 4 may determine whether a body temperature and/or a pulse exceeds a threshold value during a sport session

In particular, based on a past usage period, the CPU 4 can calculate whether a predetermined maximum load period of the usage object 100 has been exceeded based on the data generated and provided by the evaluation unit 3.

A certain maximum load duration of the usage object can be stored in advance in the CPU 4. If such a duration is exceeded, the CPU 4 can indicate to the vehicle user that the usage object 100 must be changed.

The applicant reserves the right to claim all features disclosed in the application documents as essential to the invention, provided that they are individually or in combination new compared to the prior art.

### DESCRIPTION OF THE DRAWING

FIG. 1 is a schematic representation of the system according to the present specification.

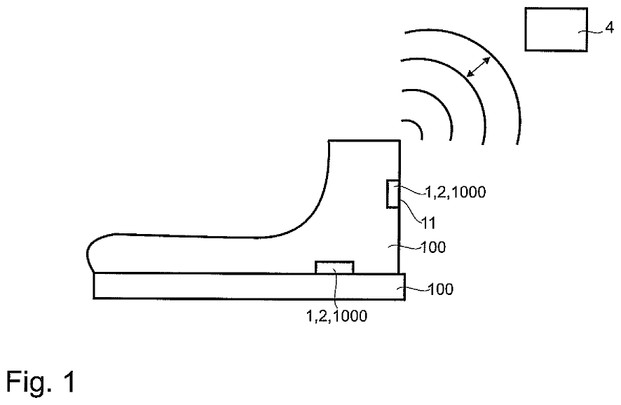

### LIST OF REFERENCE SIGNS

1 Sensor
2 Processing unit
3 Evaluation unit
4 CPU
11 Carrier material
100 Usage object
1000 Sensor arrangement
2000 Processing system

1. processing system (2000) for measuring and/or processing measured pressure and/or moisture values, for example on garments, comprising
at least one sensor (1) for measuring pressure and/or humidity,
at least one processing unit (2) which is set up and intended to control the sensor (1) and/or to store and/or process data measured by the sensor (1), and the processing unit (2) having at least one evaluation unit (3) which evaluates the data forwarded to it by the processing unit (2) and then transmits this data or a data record generated from the data to a
CPU (4), in particular wirelessly, forwards
characterized in that
at least the sensor (1), but preferably also the processing unit (2), are arranged on a flexible and bendable carrier material (11), wherein the carrier material (11) is adapted and intended to be arranged on a surface of an object of use, in particular on the surface of a piece of clothing, in particular wherein these data comprise a user behavior, movement sequences, body functions, body behavior, weight, pressure or moisture of a skin of the user, and are collected and subsequently evaluated by the CPU (4).

2. processing system (2000) according to claim 1,
characterized in that
the evaluation comprises a representation of a temporal use of the object of use, a representation of the temporal course of movement of the object of use, a representation of the temporal course of pressure and/or humidity of a skin of the user.

3. processing system (2000) according to claim 1 or 2,
characterized in that
the CPU (4), the evaluation unit (3) and/or the processing unit (2) is installed in a mobile terminal, in particular in a smartphone.

4. processing system (2000) according to claim 1 or 2,
characterized in that
the evaluation includes a proposal for action by the user for the future use of the object of use.

5. processing system (2000) according to claim 1 or 2,
characterized in that
the object of use is a textile material and/or a leather material and/or a plastic material, which is incorporated in a shoe, a shirt or any other garment of the user.

6. processing system (2000) according to claim 1 or 2,
characterized in that
the CPU (4) makes at least one utilization proposal, in particular with regard to a future temporal utilization of the utilization object, wherein the user confirms, changes and/or rejects the utilization proposal, and further wherein in the case of a change and/or a rejection of the utilization proposal by the user, the CPU (4) adapts a then changed user profile to then changed user data.

7. sensor arrangement (1000) for measuring and/or processing measured pressure and/or humidity values comprising
at least one sensor (1) for measuring pressure and/or humidity,
at least one processing unit (2) which is set up and intended to control the sensor (1) and/or to store and/or process data measured by the sensor,
characterized in that
at least the sensor (1), but preferably also the processing unit (2), are arranged on a flexible and bendable carrier material (11), wherein the carrier material (11) is adapted and intended to be arranged on or under the surface of an object of use.

8. usage object (100) for use by a user, wherein on a surface of the usage object (100) at least
a sensor (1) for measuring pressure and/or humidity is arranged,
characterized in that
at least the sensor (1) is arranged on a, in particular flexible and bendable, carrier material (11) of the object of use, the carrier material (11) being arranged on a surface of an object of use, in particular on the surface of a piece of clothing, the sensor measuring these data a user behavior, movement sequences, body functions, body behavior, weight, pressure and/or moisture of a skin of the user, the sensor (1) being set up and intended for subsequently sending these data to a CPU (4), so that these data are then subsequently evaluated by CPU (4).

9. utilization object (100) according to claim 8,
characterized in that
the sensor (1) is embedded in a shoe, in particular in a glove, or other garment to collect a movement profile, a movement intensity and/or a movement duration, which data is then sent to the CPU (4), which may be built into a game console, a smartphone and/or a PC.
